(19) United States
(12) Patent Application Publication
(10) Pub. No.: US 2022/0016200 A1
(43) Pub. Date: Jan. 20, 2022

(54) PHARMACEUTICAL COMPOSITION FOR THE TREATMENT OF CYSTIC FIBROSIS

(71) Applicant: GALENUS G.H. AG, Hergiswil (CH)

(72) Inventors: Rita DOBMEYER, Hergiswil (CH); Kurt NABER, Straubing (DE)

(21) Appl. No.: 17/312,351

(22) PCT Filed: Jun. 14, 2019
(86) PCT No.: PCT/EP2019/065782

§ 371 Date: Jun. 9, 2021

(30) Foreign Application Priority Data
Dec. 14, 2018 (EP) 18212815.7

(51) Int. Cl. A61K 38/12; A61K 38/14; A61K 31/575; A61K 9/00; A61P 31/04

(52) U.S. Cl. CPC A61K 38/12; A61K 38/14; A61P 31/04; A61K 9/0019; A61K 31/575

(57) ABSTRACT

The invention relates to a pharmaceutical composition comprising a polymyxin or a pharmaceutically acceptable salt or prodrug thereof, teicoplanin and/or fusidic acid or a pharmaceutically acceptable salt or prodrug thereof, and a pharmaceutically acceptable excipient and to ready-to-use kits for the preparation of said pharmaceutical composition.

### CROSS REFERENCE TO RELATED APPLICATIONS

This application claims priority the benefit from the priority of European patent application EP 1821815.7 filed on Dec. 14, 2018; the entire content of this application is hereby incorporated by reference.

### BACKGROUND OF THE INVENTION

### 1. Technical Field

The present invention relates to a pharmaceutical composition comprising a polymyxin or a pharmaceutically acceptable salt or prodrug thereof, teicoplanin or a pharmaceutically acceptable salt or prodrug thereof, and/or fusidic acid or a pharmaceutically acceptable salt or prodrug thereof, and a pharmaceutically acceptable excipient for the treatment of bacterial lung infections associated with cystic fibrosis.

### 2. Background Information

Infection of the airways remains the primary cause of morbidity and mortality in persons with cystic fibrosis (CF). There is an expanding spectrum of species causing infection in CF and the incidences and prevalences of infection due to specific bacterial, fungal, and viral species have changed recently.

It is estimated that approximately 30,000 persons in the United States, and an equal number elsewhere. For reasons that are incompletely understood, the alterations of airway surface liquid resulting from dysfunctional or absent Cystic fibrosis transmembrane conductance regulator (CFTR) render CF patients susceptible to chronic endobronchial infections. The associated neutrophilic inflammatory response leads to progressive lung disease and, ultimately, pulmonary failure, the primary cause of death in CF. Despite impressive advances in life expectancy in CF during the last 3 decades, the median predicted survival is approximately 37 years (Cystic Fibrosis Foundation. 2008. Patient registry 2008. Annual data report to the center directors. Cystic Fibrosis Foundation, Bethesda, Md.). The earliest bacterial pathogens include Staphylococcus aureus, Hemophilus influenzae, and Pseudomonas aeruginosa. By the end of the first decade, P. aeruginosa emerges as the predominant pathogen and remains as such until the CF patient's death.

However, according to John J. LiPuma, Clin Microbiol Rev. 2010 Apr; 23(2): 299-323 other further microbial species, including Acinetobacter spp. are involved in respiratory tract infection in CF.

Individuals that suffer from chronic CF (and progressive tissue damage due to chronic inflammation) generally experience acute exacerbations of the infection.

These chronically infected patients are generally treated with intravenous, oral and/or inhaled antibiotics, especially during these acute exacerbations of infection. Patients with chronic CF require many courses of antibiotic treatment, which may be associated with an increased risk of development of resistant microorganisms.

Antibiotic therapy of pneumonia in patients with cystic fibrosis (CF) caused by multi-drug (MDR) pathogens is very challenging. Acinetobacter non-baumanii strains play also an important role in patients with CF, which may present difficulties for therapy due to significant antimicrobial resistance (Rocha et al 2018, Species distribution, sequence types and antimicrobial resistance of Acinetobacter spp. from cystic fibrosis patients. Epidemiol Infect. 2018 Mar; 146(4):524-530. doi: 10.1017/S0950268817002849.).

Therefore, there is also a significant need for improved antibiotic regimens to treat patients with bacteria in particular Acinetobacter spp. other than A. baumanii in their pulmonary spaces.

Surprisingly it has been found that a combination of colistin and teicoplanin and/or fusidic acid is highly efficient to treat bacterial lung infections associated with cystic fibrosis.

### BRIEF SUMMARY OF THE INVENTION

Accordingly, in one embodiment of the present invention there is provided pharmaceutical composition comprising a polymyxin or a pharmaceutically acceptable salt or prodrug thereof, teicoplanin or a pharmaceutically acceptable salt or prodrug thereof, and/or fusidic acid or a pharmaceutically acceptable salt or prodrug thereof, and a pharmaceutically or a pharmaceutically acceptable salt or prodrug thereof, and/or fusidic acid or a pharmaceutically acceptable salt or prodrug thereof, and an acceptable excipient for the treatment of bacterial lung infections associated with cystic fibrosis (CF).

In a further embodiment of the invention there is provided a method for the treatment of CF which method comprises administering to a patient in need thereof pharmaceutically effective amounts of a polymyxin or a pharmaceutically acceptable salt or prodrug thereof, teicoplanin

In a further embodiment, the invention provides a kit of parts for the preparation of a pharmaceutical composition according to this invention essentially consisting of

(A) a first compartment containing a pharmaceutical composition comprising a polymyxin selected from polymyxin B and polymyxin E (colistin), or a pharmaceutically acceptable salt or prodrug thereof and a pharmaceutically acceptable excipient;

(B) a second compartment containing a pharmaceutical composition comprising teicoplanin and a pharmaceutically acceptable excipient;

(C) optionally a third compartment containing a pharmaceutical composition comprising fusidic acid or a pharmaceutically acceptable salt or prodrug thereof and a pharmaceutically acceptable excipient;

(D) optionally a leaflet describing the dosage and administration of each of the pharmaceutical compositions (A) and (B).

### DETAILED DESCRIPTION OF THE INVENTION

As used herein, the term “prodrug” relates to compounds which are quickly transformed in vivo into pharmacologically active compounds. The design of prodrugs is generally studied in Hardma et al. (Eds.), Goodman and Gilman's The Pharmacological Basis of Therapeutics, 9th ed., pages 11-16 (1996). An in-depth study is carried out in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987). In a preferred embodiment colistimethate sodium (CMS) is a prodrug of colistin.

As used herein, the term “pharmaceutically acceptable salts” includes the metal salts or the addition salts which can be used in dosage forms. For example, the pharmaceutically acceptable salts of the compounds provided herein can be acid addition salts, base addition salts or metal salts, and can be synthesized from parent compounds containing a basic or acid residue by means of conventional chemical processes. Such salts are generally prepared, for example, by reacting the free acid or base forms of these compounds with a stoichiometric amount of the suitable base or acid in water or in an organic solvent or in a mixture of both. Non-aqueous media are generally preferred, such as ether, ethyl acetate, ethanol, isopropanol, or acetonitrile. Examples of acid addition salts include mineral acid additions salts such as, for example, hydrochloride, hydrobromide, hydroiodide, sulfate, nitrate, phosphate, organic acid addition salts such as, for example, acetate, maleate, fumarate, citrate, oxalate, succinate, tartrate, malate, mandelate, methanesulfonate and p-toluenesulfonate. Examples of alkali addition salts include inorganic salts such as, for example, ammonium salts and organic alkaline salts such as, for example, diethylamine, ethylenediamine, ethanolamine, N,N-dialkylenethanolamine, triethanolamine, glutamine and basic amino acid salts. Examples of metal salts include, for example, sodium, potassium, calcium, magnesium, aluminium and lithium salts.

As used herein, the term “pharmaceutically acceptable” relates to molecular entities and compositions that are physiologically tolerable and do not normally cause an allergic reaction or a similar adverse reaction, such as gastric discomfort, dizziness and the like, when administered to humans. As used herein, the term “pharmaceutically acceptable” preferably means that it is approved by a regulatory agency of the federal or state government or listed in the US pharmacopoeia or another pharmacopoeia, generally recognized for its use in animals, preferably in mammals and more particularly in human beings.

As used herein, the term “in combination with” covers both separate and sequential administration of the anti-retroviral agent and antimicrobial agent. For example, when the agents are administered sequentially, either the teicoplanin or the polymyxin may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

As used herein, the term “teicoplanin” refers to an antibiotic used in the prophylaxis and treatment of serious infections caused by Gram-positive bacteria, including methicillin-resistant Staphylococcus aureus and vancomycin-resistant Enterococcus faecalis. It is a semisynthetic glycopeptide antibiotic with a spectrum of activity similar to vancomycin. Its mechanism of action is to inhibit bacterial cell wall synthesis. Teicoplanin is as a rule a mixture of five compounds of the following formula (I)

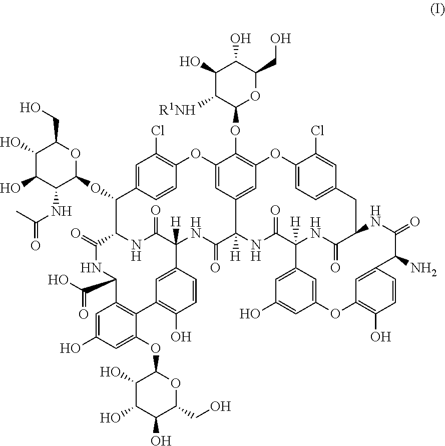

wherein

R¹ is a group selected from the formulae n-O₅H₁₁—CH═CH—(CH₂)₂—CO—, (CH₃)₂CH—(CH₂)₆—CO—, n-C₉H₁₉—CO—, C₂H₅—CH(CH₃)-(CH₂)₆-CO— and (CH₃)₂CH—(CH₂)₇—CO—

As used herein, the terms “polymyxin” or “polymyxins” relates to antibiotics, which are eventually neurotoxic and nephrotoxic, so are usually used only as a last resort if modern antibiotics are ineffective or are contraindicated. Typical uses are for infections caused by strains of multiple drug-resistant Pseudomonas aeruginosa or carbapenemase-producing Enterobacteriaceae. Polymyxins have less effect on Gram-positive organisms. Preferred polymyxins are polymyxin B and E (colistin). Polymyxin B is composed of polymyxins B1, B1-I, B2, B3, and B6. Polymyxins B1 and B2 are considered major components of formula (II)

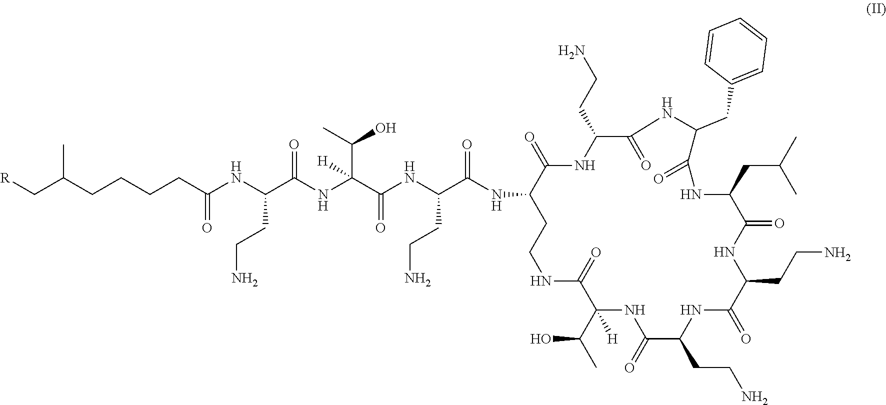

wherein R represents hydrogen (polymyxin B1) or methyl (polymyxin B2).

Polymyxin E (colistin) is a compound of formula (III)

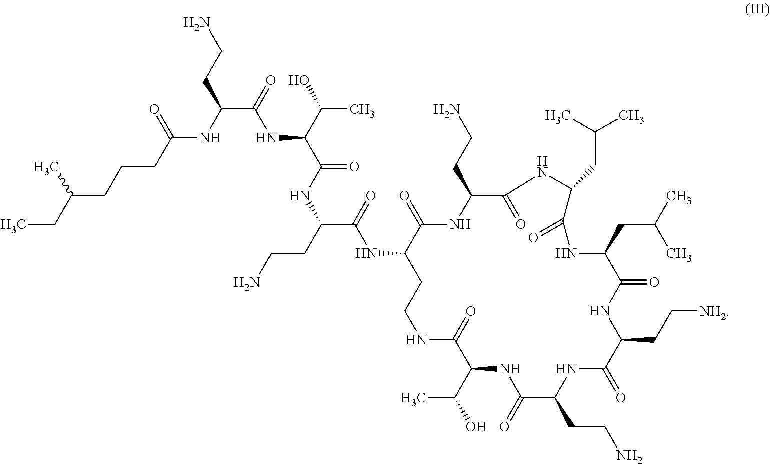 [SMILES: CCC(C)CCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

Two forms of colistin are available commercially: colistin sulfate and colistimethate sodium (colistin methanesulfonate sodium, colistin sulfomethate sodium). Colistin sulfate is cationic; colistimethate sodium is anionic. Colistin sulfate is stable, but colistimethate sodium is readily hydrolysed to a variety of methanesulfonated derivatives.

Fusidic acid is a steroid antibiotic of formula (IV)

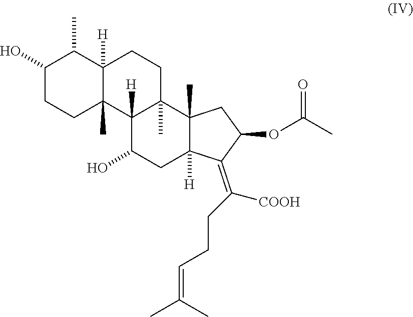

derived from the fungus Fusidium coccineum and was developed by Leo Pharma and released for clinical use in the 1960s. It has also been isolated from Mucor ramannianus and Isaria kogana. The drug is licensed for use as its sodium salt sodium fusidate, and it is approved for use under prescription in many countries. One important clinical use of fusidic acid is its activity against methicillin-resistant Staphylococcus aureus (MRSA).

According to a further embodiment of the invention, there is provided a product comprising teicoplanin and/or fusidic acid and a polymyxin selected from polymyxin B and polymyxin E (colistin), as a combined preparation for simultaneous, separate or sequential use in treating microbial infections causing cystic fibrosis (CF) particularly by killing microorganisms associated with CF, which are resistant to one component as monotherapy.

There is also provided a pharmaceutical composition comprising teicoplanin and/or fusidic acid and a polymyxin selected from polymyxin B and polymyxin E (colistin), and a pharmaceutically acceptable adjuvant, diluent or carrier.

The pharmaceutical compositions of the present invention are useful to treat microbial infections causing (CF). In particular they may be used to kill also polymyxin-resistant microorganisms associated with microbial infections. References herein to the treatment of cystic fibrosis therefore include killing polymyxin-resistant microorganisms associated with cystic fibrosis.

Preferably the pharmaceutical composition according to the invention comprises synergistically effective amounts of:
(i) a polymyxin or a pharmaceutically acceptable salt or prodrug thereof and teicoplanin or a pharmaceutically acceptable salt or prodrug thereof or
(ii) a polymyxin or a pharmaceutically acceptable salt or prodrug thereof and fusidic acid or a pharmaceutically acceptable salt or prodrug thereof.

As used herein, “kill” means a loss of viability as assessed by a lack of metabolic activity. As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection. Preferably, the term “microbial” in these contexts, means “bacterial.” As used herein, the term “bacteria” (and derivatives thereof, such as “microbial infection”) includes, but is not limited to, references to organisms (or infections due to organisms) of the following classes and specific types:

Gram-negative bacteria selected from the group consisting of Stenotrophomonas maltophilia, Acinetobacter calcoaceticus, Acinetobacter guillouiae, Acinetobacter haemolyticus, Acinetobacter johnsonii , Acinetobacter junii, Acinetobacter lwoffii, Acinetobacter nosocomialis, Acinetobacter pitii, Acinetobacter radioresistens, Acinetobacter tjernbergiae, Acinetobacter ursingi.

These Gram-negative bacteria may be accompanied by one or more of the following bacteria:

Gram-negative bacteria selected from the group consisting of Haemophilus influenza, Enterobacteriaceae species, Pseudomonas aeruginosa, Acinetobacter baumanii, Achromobacter xylosoxidans,

Gram-positive bacteria selected from the group of Staphylococcus spp., Enterococcus spp., and Streptococcus pneumoniae,

non-tuberculous mycobacteria selected from the group consisting of Mycobacterium abscessus, Mycobacterium chelonae, Mycobacterium avium complex, Mycobacterium intracellulare, Mycobacterium kansasii, Mycobacterium gordonae, Mycobacterium chelonei, and Mycobacterium fortuitum.

Preferably, the bacterial infections causing CF treated by the combinations described herein are Gram-negative infections. Preferably, the one or more Gram-negative bacterium is selected from the group consisting of Acinetobacter guillouiae, Acinetobacter haemolyticus, Acinetobacter junii, Acinetobacter lwoffii and Acinetobacter nosocomialis, which may be accompanied by Acinetobacter baumannii and/or Pseudomonas aeruginosa. The combination of the present invention is particularly beneficial in treating (multi)-drug-resistant ((M)DR) bacteria.

Further embodiments of the present invention are the following pharmaceutical compositions:

(i) which comprise teicoplanin and fusidic acid or a pharmaceutically acceptable salts or prodrugs thereof;

(ii) in which the concentration ratio of polymyxin to teicoplanin and/or fusidic acid is from 10:1 to 1:10;

(iii) which comprise 1 Million International Units (MIU) to 9 MIU, in particular 3 MIU to 4.5 MIU, corresponding to about 33 mg to 300 mg colistin base activity (CBA), in particular about 100 to 150 mg CBA of a polymyxin selected from polymyxin B and polymyxin E (colistin), or a pharmaceutically acceptable salt or prodrug thereof and 100 to 800 mg, in particular 200 to 800 mg of teicoplanin and/or 100 to 1500 mg of fusidic acid;

(iv) which are in a form suitable for parenteral administration or a solution for inhalation;

(v) wherein the pharmaceutically acceptable excipient comprises one or more fluid or semi-solid vehicles selected from the group consisting of polymers, thickeners, buffers, neutralizers, chelating agents, preservatives, surfactants, emulsifiers, antioxidants, waxes, oils, emollients, solvents and penetration enhancers; and

(vi) wherein polymyxin or a pharmaceutically acceptable salt or prodrug thereof and optionally fusidic acid or a pharmaceutically acceptable salt or prodrug thereof are administered intravenously followed by an intravenous administration of teicoplanin.

In all embodiments it is preferable that the combination therapy is synergistic as compared to the administration of the combination components taken alone. However, it should be kept in mind that although a combination such as that claimed may initially be demonstrated to be functional in treating (M)DR strains, they can then be used in treating non-resistant strains.

The active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation or insufflation administration. The most suitable route of administration may depend upon the condition and disorder of the patient. Preferably, the compositions of the invention are formulated for parenteral, inhalative or topical administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

Formulations suitable for oral administration may be presented as discrete units such as capsules, cachets or tablets (e.g. chewable tablets in particular for pediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Topical compositions, which are useful for treating disorders of the skin or of membranes accessible by digitation (such as membrane of the mouth, vagina, cervix, anus and rectum), include creams, ointments, lotions, sprays, gels and sterile aqueous solutions or suspensions. As such, topical compositions include those in which the active ingredients are dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed may contain one or more components selected from the following list: a solubilising agent or solvent (e.g. a β-cyclodextrin, such as hydroxypropyl β-cyclodextrin, or an alcohol or polyol such as ethanol, propylene glycol or glycerol); a thickening agent (e.g. hydroxymethyl cellulose, hydroxypropyl cellulose, carboxymethyl cellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (e.g. a mixture of dihydrogen phosphate and hydrogen phosphate salts, or a mixture of citric acid and a hydrogen phosphate salt). Topical formulations may also be formulated as a transdermal patch.

The most suitable route of administration may depend upon the condition and disorder of the patient.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Compositions for use according to the invention may be presented in a pack or dispenser device which may contain one or more unit dosage forms containing the active ingredients. The pack may, e.g. comprise a glass vial, a metal or plastic foil, such as a blister pack. Where the compositions are intended for administration as two separate compositions these may be presented in the form of a twin pack or a kit.

Compositions for inhalation will be administered by an inhaler (or puffer), which is a medical device used for delivering medication into the body via the lungs. Preferred inhalers are pressurized metered-dose inhalers (MDI), which are made up of 3 standard components—a metal canister, plastic actuator, and a metering valve, dry powder inhalers, which release a metered or device-measured dose of powdered medication and mechanically pressurized inhalers such as the Soft Mist Inhaler Respimat®.

Pharmaceutical compositions may also be prescribed to the patient in kit or “patient packs” containing the whole course of treatment in a single package, usually a blister pack or a pack of glass vials. Patient packs have an advantage over traditional prescriptions, where a pharmacist divides a patients' supply of a pharmaceutical from a bulk supply, in that the patient or the treating health professional always has access to the package insert contained in the patient pack, normally missing in traditional prescriptions. The inclusion of the package insert has been shown to improve patient compliance with the physician's instructions. The administration of the combination of the invention by means of a single patient pack, or patient packs of each composition, including a package insert directing the patient to the correct use of the invention is a desirable feature of this invention.

According to a further embodiment of the present invention there is provided a kit comprising at least one active ingredient of the combination according to the invention and an information insert containing directions on the use of the combination of the invention.

In a first embodiment the kits according to the invention comprise:

(A) a first compartment containing a pharmaceutical composition comprising 1 MIU to 9 MIU, of a polymyxin selected from polymyxin B and polymyxin E (colistin), or a pharmaceutically acceptable salt or prodrug thereof and a pharmaceutically acceptable excipient; and

(B) a second compartment containing a pharmaceutical composition comprising 100 to 800 mg of teicoplanin and a pharmaceutically acceptable excipient and/or 100 to 1500 mg fusidic acid a pharmaceutically acceptable excipient.

Furthermore, the invention relates to kits, wherein the components (A) and/or (B) consist of the sub-compartments

(A1) containing a polymyxin selected from polymyxin B and polymyxin E (colistin), or a pharmaceutically acceptable salt or prodrug thereof and optionally a solid carrier;

(A2) containing an aqueous diluent for preparation of an injectable solution of said polymyxin; and/or

(B1) containing teicoplanin and/or fusidic acid and optionally a solid carrier;

(B2) containing an aqueous diluent for preparation of an injectable solution of teicoplanin.

Suitable dosages and formulations for the administration of colistin are described in the product label for Colomycin® which can be found at https://www.medicines.org.uk/emc/medicine/1590.

Suitable dosages and formulations for the inhalative administration of colistin are described in the SPC for colistimethate sodium Colobreathe® which can be found at http://www.ema.europa.eu/docs/en_GB/document_library/EPAR_-_Product_Information/human/001225/WC500123690.pdf.

Suitable dosages and formulations for the administration of teicoplanin are described in the product label for Targocid® 400 mg powder for solution for injection/infusion or oral solution which can be found at https://www.medicines.org.uk/emc/medicine/27321.

Suitable dosages and formulations for the administration of sodium fusidate are described in the product label for Fucidin® tablets which can be found at https://www.medicines.org.uk/emc/medicine/2448#PRODUCTINFO.

The route of administration and dosage of polymyxin B and polymyxin E (colistin) is generally determined by the administering physician. Typically, polymyxin B and polymyxin E (colistin) is administered by topical, intramuscular, intravenous, intrathecal, inhalative or ophthalmic routes depending on the nature of the bacterial infection.

The administration of the combination of the invention by means of a single patient pack, or patient packs of each composition, including a package insert directing the patient to the correct use of the invention is a desirable feature of this invention. According to a further embodiment of the present invention there is provided a patient pack comprising at least one active ingredient of the combination according to the invention and an information insert containing directions on the use of the combination of the invention.

The amount of active ingredients required for use in treatment will vary with the nature of the condition being treated and the age and condition of the patient, and will ultimately be at the discretion of the attendant physician or veterinarian. In general however, doses of the combined active ingredients employed for adult human treatment will typically be in the range of 0.02 to 5000 mg per day, preferably Ito 1500 mg per day. The desired dose may conveniently be presented in a single dose or as divided doses administered at appropriate intervals, e.g. as two, three, four or more sub-doses per day.

Because of its very long terminal half-life of teicoplanin with a mean (±SD) of 157 (±93) hours (h) or with a median (range) of 168 (111-278) h the first 3-6 doses of 400 mg will be administered every 12 h, followed by 400 mg every 24. Preferably these doses of teicoplanin will be provided in form of a powder of teicoplanin with a carrier, preferably a salt such as sodium chloride for solution for injection or infusion.

Accordingly, the treatment kit also includes one combination of colistin 4.5 MIU, e.g. given as colistimethate sodium (CMS), a ‘prodrug’ that is converted to colistin in the body, plus teicoplanin 400 mg to be administered intravenously (IV) the first 3-6 doses every 12 h (bid). Thereafter a combination of colistin 4.5 MIU plus teicoplanin 200 mg to be administered IV every 12 h (bid). By this way the same colistin dose with half of the teicoplanin dose is further administered during steady state bid. If a lower dosage of colistin is appropriate, e.g. 3 MIU bid for treatment of complicated UTI, then a kit will be provided with vials containing colistin 3 MIU, e.g. as CMS, and teicoplanin 400 mg and 200 mg, respectively.

In case of severely ill patients where a loading dose of colistin upto 9 MIU is recommended 2 vials containing each colistin 4.5 MIU plus teicoplanin 400 mg (total colistin 9 MIU plus teicoplanin 800 mg) can be administered, because teicoplanin 800 mg as first loading dose is recommended in severe infections. Since the kit also contains vials with two different amounts of colistin (4.5 MIU and 3 MIU) combined either with teicoplanin 400 mg and 200 mg a more individual loading dose for colistin and teicoplanin is possible.

Since both, colistin and teicoplanin, are mainly excreted through the kidneys, in case of renal insufficiency the dosage reduction following the first normal dose can be made according to the degree of the individual renal insufficiency proportionally at about the same manner as recommended for colistin.

nd/or one or more other anti-bacterials including anti-tubercular compounds such as azithromycin, ceftriaxone, cefixime, ciprofloxacin, spectinomycin and vancomycin and/or other vitamins including vitamin E. If the other antibacterial is a β-lactam then a β-lactamase inhibitor may also be employed.

The pharmaceutical formulations according to the invention comprising a polymyxin, teicoplanin and/or fusidic acid may be advantageously combined with further adjuvants and medications.

Inhaled therapy with other antibiotics such as tobramycin and aztreonam may be additionally administered Inhaled levofloxacin may be additionally used to treat Pseudomonas aeruginosa. The early management of Pseudomonas aeruginosa infection is easier and better, using nebulized antibiotics with or without oral antibiotics may sustain its eradication up to 2 years.

Oral antibiotics such as ciprofloxacin or azithromycin may be additionally given to help prevent infection or to control ongoing infection. However, the aminoglycoside antibiotics such as tobramycin can cause hearing loss, damage to the balance system in the inner ear or kidney failure with long-term use.

The co-administration of vitamin C may be beneficial in view of its nephron-protective effect.

In addition, vitamin D, in particular vitamin D₃ or calcitriol may enhance the efficiency of the pharmaceutical composition of this invention.

Mucolytics that help loosen secretions such as acetylcysteine, ambroxol, bromhexine, carbocisteine, domiodol, dornase alfa, eprazinone, erdosteine, letosteine, mannitol, mesna, neltenexine, sobrerol, stepronin and tiopronin may be advantageously co-admnistered.

The same applies to silver nanoparticles, which may be added to the inhalative formulation of the invention.

The invention now being generally described, will be more readily understood by reference to the following Examples, which are included merely for purposes of illustration of certain aspects and embodiments of the present invention, and are not intended to limit the invention.

### EXAMPLES

### Example 1 In-Vitro Synergistic Antibacterial Activity of Teicoplanin and Colistin

Checkerboard analysis to determine minimal inhibitory concentration (MIC)/fractional inhibitory concentration (FIC) and minimal bacterial concentration (MBC)/fractional bacterial concentration (FBC) values of colistin sulphate (CS) plus teicoplanin (TP) for 6 Gram-negative strains with a bacterial load 5(1-10)*10⁵ CFU/ml in a cation-adjusted Mueller-Hinton broth (CAMHB).
a. Antimicrobial stock solutions were prepared using sterile distilled water
b. CS was added along the ordinate; TP was added along the abscissa to 96 well, flat bottom, polystyrene panels with no treatment. Colistin alone was tested in triplicates.
c. Serial 2-fold dilutions starting with 640 μg/ml CS and 640 μg/ml TP were made; 15 μl volume of the serial dilutions were added for each agent to the appropriate wells; final volume at 30 μl
d. For each agent tested alone an additional 15 μl media was added to the well. 30 μl were added in control wells where none of the agent was tested. The negative control well contains 150 μl media without any additions
e. From an overnight bacterial culture in tryptic soy broth, 100 μL was added to 10 mL of fresh tryptic soy broth. This was then incubated at 37° C. for 1.5 hour at 180 rpm.
f. Bacteria in 120 μl CAMHB were added to each well (end concentration 5(1-10)*10⁵ CFU/ml)
g. Plates were incubated at 37° C. in an ambient air incubator for 24 h
h. Turbidity at OD₆₀₀ is determined with a plate photometer
i. By using a one-time inoculator 3 μl from each well of the 96-well plate were transferred to three blood agar plates. The plates were incubated over night at 37° C. in an ambient air incubator
j. MIC values are defined as lowest concentration which restricts bacterial growth to OD₆₀₀ ≤0.1
k. MBC values were determined by visual reading the lowest concentration which inhibits 99.9% of the growth of bacteria in the wells
l. Determination of the fractional inhibitory concentration (FIC) index and fractional bactericidal concentration (FBC) index and characterization of antimicrobial interactions was assessed

FIC calculation was carried out as: (MIC of drug A, tested in combination)/(MIC of drug A, tested alone)+(MIC of drug B, tested in combination)/(MIC of drug B, tested alone). FBC calculation was carried out just as well

Interactive categories were calculated from checkerboard analysis using MIC results from testing each agent alone and MIC results from the combination wells adjacent to the wells with growth in them

a. Synergy; FIC/FBC≤0.5

b. Indifference; FIC/FBC>0.5 to<4.0

c. Antagonism; FIC/FBC≥4.0

Organisms:

a. A. junii 1391597 (IHMA), colistin resistant

b. A. nosocomialis 1461911 (IHMA), colistin resistant

c. A. haemolyticus 1655843 (IHMA), colistin resistant

d. A. guillouiae 1285286 (IHMA), colistin resistant

e. Stenotrophomonas maltophilia 1221783 (IHMA), colistin susceptible

f. S. maltophilia 1237289 (IHMA), colistin resistant

The FIC/FBC of the test strains for TEC and CS are shown in the following Tables I and II:

TABLE I
FIC indices from checkerboard titration synergy testing in CAMHB with inoculum 5*10⁵ CFU/ml

| | MIC [mg/l] | | MIC results for combination wells (FD/CS) and FIC indices (ΣFIC) | | | | | | | | ΣFIC | | |
|---|---|---|---|---|---|---|---|---|---|---|---|---|---|
| | TP | CS | result | ΣFIC | result | ΣFIC | result | ΣFIC | result | ΣFIC | Min | mean | Max |
| A. junii | >64 | 2 | 4/1 | 0.531 | 16/0.5 | 0.375 | 64/0.25 | 0.625 | | | 0.375 | 0.510 | 0.625 |
| 1397597 | | | | | | | | | | | | | |
| A. nosocomialis | >64 | 4 | 8/2 | 0.563 | 32/1 | 0.5 | 32/0.5 | 0.375 | | | 0.375 | 0.479 | 0.563 |
| 1461911 | | | | | | | | | | | | | |
| A. haemolyticus | >64 | >16 | 16/16 | 0.625 | 16/8 | 0.375 | 32/4 | 0.375 | 64/2 | 0.563 | 0.375 | 0.485 | 0.563 |
| 1655843 | | | | | | | | | | | | | |
| A. guillouiae | >64 | 2 | 4/1 | 0.531 | 8/0.5 | 0.313 | 64/0.25 | 0.625 | | | 0.313 | 0.490 | 0.625 |
| 1285286 | | | | | | | | | | | | | |
| S. maltophilia | >64 | 0.25 | 0.25/0.25 | 1.002 | | | | | | | 1.002 | | |
| 1221783 | | | | | | | | | | | | | |
| S. maltophilia | >64 | 0.5 | 32/0.25 | 0.75 | (1/4) | (0.508) | (2/2) | (0.266) | (32/1) | (0.375) | 0.75 | 0.75 | 0.75 |
| 1237289* | (>64) | (8) | | | | | | | | | (0.266) | (0.383) | (0.508) |

*S.maltophilia 1237289 grew very slow, therefor MIC/FIC values after 48 h growth are added in brackets

TABLE II
FBC indices from checkerboard titration synergy testing in CAMHB with inoculum 5*10⁵ CFU/ml

| | MBC [mg/l] | | MIC results for combination wells (FD/CS) and FBC indices (ΣFBC) | | | | | | | | ΣFBC | | |
|---|---|---|---|---|---|---|---|---|---|---|---|---|---|
| | TP | CS | result | ΣFBC | result | ΣFBC | result | ΣFBC | result | ΣFBC | Min | mean | Max |
| A. junii | >64 | 8 | 0.25/4 | 0.502 | 0.5/2 | 0.254 | 8/1 | 0.188 | 32/0.5 | 0.313 | 0.188 | 0.314 | 0.502 |
| A. nosocomialis | >64 | 8 | 8/4 | 0.563 | 32/2 | 0.5 | 32/1 | 0.375 | 64/0.5 | 0.563 | 0.375 | 0.500 | 0.563 |
| A. haemolyticus | >64 | >16 | 64/16 | 1 | 64/8 | 0.75 | 64/4 | 0.625 | | | 0.625 | 0.792 | 1 |
| A. guillouiae | >64 | 4 | 0.5/2 | 0.504 | 8/1 | 0.313 | 32/0.5 | 0.375 | | | 0.313 | 0.396 | 0.504 |
| S. maltophilia | >64 | 2 | 32/1 | 0.75 | 32/0.5 | 0.5 | 32/0.25 | 0.375 | | | 0.375 | 0.542 | 0.75 |
| S. maltophilia | >64 | 8 | 1/4 | 0.508 | 2/2 | 0.266 | 32/1 | 0.375 | | | 0.266 | 0.383 | 0.508 |

Conclusions

MIC values of colistin were ≥4 mg/l for Acinetobacter nosocomialis 1461911 and A. haemolyticus 1655843 classifying them as colistin resistant according to EUCAST breakpoints. MIC values for A. junii 1397597 and A. guillouiae 1655843 were 2 mg/l. Since EUCAST breakpoints are ≤2 mg/l=sensitive and >2 mg/l, both strains are difficult to classify. After 24 h growth both S. maltophilia strains showed a MIC value of colistin ≤0.5 mg/l, classifying them as colistin sensitive. However, S. maltophilia 1237289 grew very slowly. After 48 h incubation, this strain had an MIC value of 8 mg/l. Checkerboard assays showed synergy between TP and CS for the A. junii 1397597, A. haemolyticus 1655843, A. guillouiae 1655843 based on both, inhibitory and bactericidal levels. For A. nosocomialis 1461911, S. maltophilia 1237289 and S. maltophilia 1221783 synergy could be shown only for one, either FIC or FBC values.

### Example 2 Treatment of Cystic Fibrosis

Methicillin-resistant Staphylococcus aureus (MRSA) is a bacterial pathogen in patients with cystic fibrosis (CF) although its clinical effects can be variable. In a three-step decolonization protocol for MRSA 15 paediatric patients are treated during a long follow-up period where 50% are successfully decolonized following one five-day course of i.v. Colistin and fusidic acid. To increase the success rate the patients are to receive a five-day intravenous teicoplanin treatment to clear MRSA colonization to 90-100%.

### Example 3 In-Vitro Synergistic Antibacterial Activity of Fusidic Acid and Colistin

Methods:

Checkerboard assays between fusidic acid (FD) and colistin sulfate (CS) with a bacterial load 5(1-10)*10⁵ CFU/ml in cationic adapted Mueller Hinton broth (CAMHB).
a. Antimicrobial stock solutions were prepared using sterile distilled water;
b. CS was added along the ordinate; FD was added along the abscissa to 96 well, flat bottom, polystyrene panels with no treatment. Colistin alone was tested in triplicates;
c. Serial 2-fold dilutions starting with 640 1.1 g/ml CS and 640 μg/ml FD were made; 15 μl volume of the serial dilutions were added for each agent to the appropriate wells—final volume at 30 μl;
d. For each agent tested alone an additional 15 μl media was added to the well. 30 μl were added in control wells where none of the agent was tested. The negative control well contains 150 μl media without any additions;
e. From an overnight bacterial culture in tryptic soy broth, 100 μL was added to 10 mL of fresh tryptic soy broth. This was then incubated at 37° C. for 1.5 hour at 180 rpm;
f. Bacteria in 120 μl CAMHB were added to each well (end concentration 5(1-10)*10⁵ CFU/ml);
g. Plates were incubated at 37° C. in an ambient air incubator for 24 h
h. Turbidity at OD₆₀₀ is determined with a plate photometer
i. By using a one-time inoculator 3 μl from each well of the 96-well plate were transferred to three blood agar plates. The plates were incubated over night at 37° C. in an ambient air incubator;
j. MIC values are defined as lowest concentration which restricts bacterial growth to OD₆₀₀≤0.1;
k. MBC values were determined by visual reading the lowest concentration which inhibits 99.9% of the growth of bacteria in the wells;
l. Determination of the fractional inhibitory concentration (FIC) index and fractional bactericidal concentration (FBC) index and characterization of antimicrobial interactions was assessed;

FIC calculation was carried out as: (MIC of drug A, tested in combination)/(MIC of drug A, tested alone)+(MIC of drug B, tested in combination)/(MIC of drug B, tested alone). FBC calculation was carried out just as well;

Interactive categories were calculated from checkerboard analysis using MIC results from testing each agent alone and MIC results from the combination wells adjacent to the wells with growth in them.

Determination of the fractional inhibitory concentration (FIC) index and fractional bactericidal concentration (FBC) index and characterization of antimicrobial interactions was assessed:

(i) Synergy; FIC/FBC≤0.5

(ii) Indifference; FIC/FBC>0.5 to <4.0

(iii) Antagonism; FIC/FBC≥4.0

Organisms (Colistin CS MIC/MBC mg/L):

a. Acinetobacter junii 1391597 (IHMA), colistin resistant (CS 2/2-2/8)

b. Acinetobacter nosocomialis 1461911 (IHMA), colistin resistant (3xCS 4/8)

c. Acinetobacter haemolyticus 1655843 (IHMA), colistin resistant (3xCS>16/>16-64/>64)

d. Acinetobacter guillouiae 1285286 (IHMA), colistin resistant (2xCS 2/4)

e. Stenotrophomonas maltophilia 1237289 (IHMA), colistin resistant (CS 2/4-4/8-8/8)

The FIC/FBC of the test strains for FD and CS are shown in the following Tables III and IV:

TABLE III
FIC indices from checkerboard titration synergy testing in CAMHB with inoculum 1*10⁶ CFU/ml

| | MIC [mg/1l] | | MIC results for combination wells (FD/CS) and FIC indices (ΣFIC) | | | | | | | | ΣFIC | | |
|---|---|---|---|---|---|---|---|---|---|---|---|---|---|
| | FD | CS | result | ΣFIC | result | ΣFIC | result | ΣFIC | result | ΣFIC | Min | mean | Max |
| A. junii | 32 | 2 | 1/1 | 0.531 | 4/0.5 | 0.375 | | | | | 0.375 | 0.453 | 0.531 |
| 1397597 | | | | | | | | | | | | | |
| A. nosocomialis | >64 | 4 | 2/2 | 0.516 | 8/1 | 0.313 | 32/0.5 | 0.375 | 64/0.25 | 0.563 | 0.313 | 0.442 | 0.563 |
| 1461911 | | | | | | | | | | | | | |
| A. haemolyticus | 32 | 64 | 0.25/8 | 0.133 | 0.5/4 | 0.078 | 1/2 | 0.063 | 4/1 | 0.141 | 0.063 | 0.104 | 0.141 |
| 1655843 | | | | | | | | | | | | | |
| A. guillouiae | 16 | 2 | 0.25/1 | 0.516 | 2/0.5 | 0.375 | | | | | 0.375 | 0.446 | 0.516 |
| 1285286 | | | | | | | | | | | | | |
| S. maltophilia | 64 | 0.5 | 0.25/0.5 | 1.004 | | | | | | | 1.004 | 1.004 | 1.004 |
| 1221783 | | | | | | | | | | | | | |
| S. maltophilia | >64 | 1 | 0.25/1 | 1.002 | (2/2) | (0.516) | (32/1) | (0-5) | | | 1.002 | 1.002 | 1.002 |
| 1237289* | (>64) | (4) | | | | | | | | | (0-5) | (0.508) | (0.516) |

*S.maltophilia 1237289 grew very slow, therefor MIC/FIC values after 48 h growth are added in brackets

TABLE IV
FBC indices from checkerboard titration synergy testing in CAMHB with inoculum 1*10⁶ CFU/ml

| | MBC [mg/l] | | MIC results for combination wells (FD/CS) and FBC indices (ΣFBC) | | | | | | | | ΣFBC | | |
|---|---|---|---|---|---|---|---|---|---|---|---|---|---|
| | FD | CS | result | ΣFBC | result | ΣFBC | result | ΣFBC | result | ΣFBC | Min | mean | Max |
| A. junii | 64 | 2 | 8/1 | 0.625 | | | | | | | 0.625 | 0.625 | 0.625 |
| 1397597 | | | | | | | | | | | | | |
| A. nosocomialis | >64 | 8 | 32/2 | 0.5 | 64/0.5 | 0.563 | | | | | 0.5 | 0.532 | 0.563 |
| 1461911 | | | | | | | | | | | | | |
| A. haemolyticus | 64 | >64 | 1/64 | 0.516 | 2/32 | 0.281 | 8/8 | 0.188 | 32/4 | 0.531 | 0.188 | 0.379 | 0.531 |
| 1655843 | | | | | | | | | | | | | |
| A. guillouiae | >64 | 4 | 1/2 | 0.508 | 32/1 | 0.5 | 64/0.5 | 0.625 | | | 0.5 | 0.542 | 0.625 |

Results:

Checkerboard assays showed synergy between fusidic acid (FD) and colistin sulfate (CS) for Acinetobacter nosocomialis 1461911, Acinetobacter haemolyticus 1655843, Acinetobacter guillouiae 1655843 based on both, inhibitory (FIC) and bactericidal (FBC) levels. For A. junii 1397597 and Stenotrophomonas maltophilia 1237289 synergy could be shown only for one, either FIC or FBC values.

### Example 4 Comparison of Colistin Monotherapy with Colistin Combination Therapy with Teicoplanin or Fusidic Acid in a Pneumonic Mouse Model

As Example 1 and Example 3 show, the combination of colistin and teicoplanin or fusidic acid is in vitro synergistic against several A. non-baumanii strains, a pneumonic mouse model is used to demonstrate this synergistic effect also in vivo.

Material and Methods

One colistin susceptible (MIC 1 mg/L) A. nosocomialis strain (1204194 IHMA) and one colistin resistant (MIC 4 mg/L) A. nosocomialis strain (1461911 IHMA) are used. For the colistin resistant strain in vitro by checkerboard test synergy between colistin sulphate and teicoplanin or fusidic acid is demonstrated (FIC<0.5). Fresh brain heart infusion broth (Merck, Darmstadt, Germany) bacterial cultures, in an aerobic atmosphere in the logarithmic growth phase (4-5 hours) at 37° C., are adjusted to a concentration of 1.0×10⁶ colony forming units (CFU)/mL, as verified by both spectrophotometry (OD600 0.01-0.02 nm) and colony counting.

Mice

A pneumonic mouse model was used. 8- to 12-week-old, specific-pathogen free, male or female BALB/c mice, with 25-35 g in weight, are used for the study Animals have free access to food and water except during experimental procedures. All studies are performed in accordance with the ethical guidelines for the care and use of laboratory animals, and the protocol is approved by an independent ethical commission.

Acinetobacter Nosocomialis Inoculation

For inoculation, fresh inocula are prepared for each experiment from frozen stocks of the two A. nosocomialis isolates (1204194 and 1461911 IHMA). Broth cultures of freshly plated A. nosocomialis bacteria are grown to logarithmic phase overnight to an absorbance of 0.3 at 630 nm and diluted to 10⁷ CFU/ml in saline. Mice are anesthetized by intraperitoneal (i.p.) injection of 12.5 mg/kg (5 μl) xylazine and 80 mg/kg (25 μl) ketamine and then inoculated intranasally with 0.05 ml of this bacterial suspension.

Twenty-four mice are assigned to eight groups (3 mice in each group) using four treatment modalities: saline, colistin sulphate, colistin sulphate combination with teicoplanin, colistin sulphate combination with fusidic acid.

Antibiotic Therapy

24 hours after intra-tracheal inoculation the mice receive one intramuscular thigh injection of saline or one dose of colistin sulphate (8 mg/kg) or one dose of a combination therapy with colistin sulphate (8 mg/kg) and teicoplanin (20 mg/kg) or one dose of a combination therapy with colistin sulphate (8 mg/kg) and fusidic acid (50 mg/kg).

Conventional Bacterial Culture of Pneumonic Mice Lung

Mice, which survive, are sacrificed on day 3 post inoculation. The lung (about 0.36 g) is removed and segmented and then homogenized under the sterile condition. Next, 1 ml saline is added to the homogenized tissue, and 100 μ1 was cultured on Muller-Hinton agar (MHA) and then incubated at 37° C. for one day. One part of tissue is removed for qRT-PCR as described by Hassannejad N, Bahador A, Rudbari N H, Modarressi M H, Parivar K. Comparison of OmpA Gene-Targeted Real-Time PCR with the Conventional Culture Method for Detection of Acinetobacter baumanii in Pneumonic BALB/c Mice. Iranian Biomedical Journal 2019; 23 (2): 159-164.

Results

None of the two groups of mice treated with saline survive until day 3. Of the three mice having received the colistin susceptible A. nosocomialis strain and treated with colistin monotherapy only 2/3 of the mice survive and of the three mice having received the colistin resistant A. nosocomialis strain and treated with colistin monotherapy 0/3 of the mice survive until day 3. Of the two groups, one having received the colistin susceptible and one the resistant A. nosocomialis strain, all 6 mice treated with the combination therapy of colistin and teicoplanin survive until day 3. Of the two groups, one having received the colistin susceptible and one the resistant A. nosocomialis strain, all 6 mice treated with the combination therapy of colistin and fulvic acid survive until day 3.

The lung tissue of the two mice having received the colistin susceptible A. nosocomialis strain survive with colistin monotherapy shows significantly higher numbers of A. nosocomialis in the lung tissue confirmed by qRT-PCR than all the mice treated with the combination of colistin sulphate and teicoplanin or colistin sulphate and fusidic acid.

Conclusion

In a pneumonic mouse model it can be shown that the combination therapy with colistin and teicoplanin or colistin and fulvic acid is more effective than colistin monotherapy against a colistin susceptible and a colistin resistant Acinetobacter non-baumanii strain (A. nosocomialis). Therefore, especially in patients with cystic fibrosis with pneumonia caused by MDR Acinetobacter species whether colistin susceptible or resistant, a combination therapy of colistin and teicoplanin or colistin and fulvic acid should be tested in well-designed clinical studies.

1.-15. (canceled)

16. A method for the treatment of a bacterial lung infection associated with cystic fibrosis in a patient in need thereof comprising administering to the patient in need thereof:
(a) a polymyxin or a pharmaceutically acceptable salt or prodrug thereof; and
(b) at least one additional agent chosen from teicoplanin or a pharmaceutically acceptable salt or prodrug thereof, fusidic acid or a pharmaceutically acceptable salt or prodrug thereof, or combinations thereof.

17. The method according to claim 16, wherein the polymyxin is chosen from polymyxin B or polymyxin E (colistin).

18. The method according to claim 16, wherein the bacterial lung infection associated with cystic fibrosis is caused by one or more Gram-negative bacteria selected from the group consisting of Stenotrophomonas maltophilia, Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingi, or combinations thereof.

19. The method according to claim 18, wherein the bacterial lung infection associated with cystic fibrosis is additionally caused by:
one or more Gram-negative bacteria selected from the group consisting of Haemophilus influenza, Enterobacteriaceae species, Pseudomonas aeruginosa, Acinetobacter baumanii, Achromobacter xylosoxidans, or combinations thereof;
one or more Gram-positive bacteria selected from Staphylococcus spp.; and/or
one or more nontuberculous mycobacteria selected from the group consisting of Mycobacterium abscessus, Mycobacterium chelonae, Mycobacterium avium complex, Mycobacterium intracellulare, Mycobacterium kansasii, Mycobacterium gordonae, Mycobacterium chelonei, Mycobacterium fortuitum, or combinations thereof.

20. The method according to claim 19, wherein the one or more Gram-positive bacteria are chosen from methicillin-resistant Staphylococcus aureus (MRSA), Enterococcus spp., Streptococcus pneumoniae, or combinations thereof.

21. The method according to claim 16, wherein:
synergistically effective amounts of the polymyxin or a pharmaceutically acceptable salt or prodrug thereof and teicoplanin or a pharmaceutically acceptable salt or prodrug thereof are administered to the patient as a combination in a pharmaceutical composition; or
(ii) synergistically effective amounts the polymyxin or a pharmaceutically acceptable salt or prodrug thereof and fusidic acid or a pharmaceutically acceptable salt or prodrug thereof are administered to the patient as a combination in a pharmaceutical composition.

22. The method according to claim 16, wherein the concentration ratio of (a) the polymyxin or a pharmaceutically acceptable salt or prodrug thereof to (b) at least one additional agent chosen from teicoplanin or a pharmaceutically acceptable salt or prodrug thereof, fusidic acid or a pharmaceutically acceptable salt or prodrug thereof, or combinations thereof ranges from 10:1 to 1:10.

23. The method according to claim 16, wherein 1 MIU to 9 MIU of the polymyxin or a pharmaceutically acceptable salt or prodrug thereof is administered to the patient in combination with 100 mg to 800 mg of teicoplanin or a pharmaceutically acceptable salt or prodrug thereof, and/or 100 mg to 1500 mg of fusidic acid or a pharmaceutically acceptable salt or prodrug thereof.

24. The method according to claim 16, wherein 3 MIU to 4.5 MIU of the polymyxin or a pharmaceutically acceptable salt or prodrug thereof is administered to the patient.

25. The method according to claim 16, wherein 200 to 800 mg of teicoplanin or a pharmaceutically acceptable salt or prodrug thereof and/or 100 to 1500 mg of fusidic acid or a pharmaceutically acceptable salt or prodrug thereof is administered to the patient.

26. The method according to claim 16, comprising administering to a patient a pharmaceutical composition comprising:
(a) a polymyxin or a pharmaceutically acceptable salt or prodrug thereof; and
(b) at least one additional agent chosen from teicoplanin or a pharmaceutically acceptable salt or prodrug thereof, fusidic acid or a pharmaceutically acceptable salt or prodrug thereof, or combinations thereof;
wherein the pharmaceutical composition further comprises a pharmaceutically acceptable excipient.

27. The method according to claim 26, wherein the pharmaceutical composition is administered to the patient through parenteral administration or inhalation.

28. The method according to claim 26, wherein the pharmaceutically acceptable excipient is chosen from polymers, thickeners, buffers, neutralizers, chelating agents, preservatives, surfactants, emulsifiers, antioxidants, waxes, oils, emollients, solvents, penetration enhancers, or combinations thereof.

29. The method according to claim 16, wherein the polymyxin or a pharmaceutically acceptable salt or prodrug thereof is administered intravenously to the patient, followed by an intravenous administration of teicoplanin or a pharmaceutically acceptable salt or prodrug thereof.

30. The method according to claim 16, wherein the polymyxin or a pharmaceutically acceptable salt or prodrug thereof and fusidic acid or a pharmaceutically acceptable salt or prodrug thereof are administered intravenously to the patient, followed by an intravenous administration of teicoplanin or a pharmaceutically acceptable salt or prodrug thereof.

31. A method for the treatment of a patient suffering from a bacterial infection, comprising administering to the patient synergistically effective amounts of
(a) a polymyxin selected from polymyxin B or a pharmaceutically acceptable salt or prodrug thereof, polymyxin E (colistin) or a pharmaceutically acceptable salt or prodrug thereof, or combinations thereof; and
(b) fusidic acid or a pharmaceutically acceptable salt or prodrug thereof, wherein the bacterial infections are caused by one or more Gram-negative bacteria selected from the group consisting of Stenotrophomonas maltophilia, Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingi, or combinations thereof.
